(19) United States
(12) Patent Application Publication
(10) Pub. No.: US 2024/0307189 A1
(43) Pub. Date: Sep. 19, 2024

(54) INTERSPINOUS SPACER WITH A RANGE OF DEPLOYMENT POSITIONS AND METHODS AND SYSTEMS

(71) Applicant: Boston Scientific Neuromodulation Corporation, Valencia, CA (US)

(72) Inventor: Zdzislaw Bernard Malinowski, Castaic, CA (US)

(21) Appl. No.: 18/602,968

(22) Filed: Mar. 12, 2024

Related U.S. Application Data
(60) Provisional application No. 63/452,249, filed on Mar. 15, 2023.

(51) Int. Cl. A61F 2/44; A61B 17/88; A61F 2/30; A61F 2/46

(52) U.S. Cl. CPC A61F 2/4425; A61B 17/8888; A61F 2/4611; A61F 2002/30405; A61F 2002/30494; A61F 2002/30495; A61F 2002/30528; A61F 2002/4615

(57) ABSTRACT

An interspinous spacer includes a body having a channel and at least one slot; an arm actuator defining a threaded channel; an actuator screw including a shaft with a threaded distal portion partially disposed in the channel of the body and the threaded channel of the arm actuator; a first pin arranged to move along the slot of the body; a second pin; and first and second arms, each having a coupling extension that defines a pin opening and a curved track. The first and second arms are coupled to the body by the second pin extending through the curved tracks and further coupled to the body and the actuator arm by the first pin extending through the pin openings. The first and second arms rotate among different deployment positions according to the curved track in response to longitudinal movement of the actuator arm as the actuator screw is rotated.

### CROSS-REFERENCE TO RELATED APPLICATIONS

This application claims the benefit under 35 U.S.C. § 119(e) of U.S. Provisional Patent Application Ser. No. 63/452,249, filed Mar. 15, 2023, which is incorporated herein by reference.

### FIELD

The present invention is directed to the area of interspinous spacers for deployment between adjacent spinous processes. The present invention is also directed to systems and methods for utilizing the interspinous spacer.

### BACKGROUND

With spinal stenosis, the spinal canal narrows and pinches the spinal cord and nerves, causing pain in the back and legs. Typically, with age, a person's ligaments may thicken, intervertebral discs may deteriorate, or facet joints may break down. The conditions can contribute to the narrowing of the spinal canal. Injury, heredity, arthritis, changes in blood flow, and other causes may also contribute to spinal stenosis.

Various treatments of the spine have been proposed or used including medications, surgical techniques, and implantable devices that alleviate and reduce pain associated with the back. In one surgical technique. a spacer is implanted between adjacent spinous processes of a patient's spine. The implanted spacer opens the spinal canal, maintains the desired distance between vertebral body segments, and, as a result, avoids or reduces impingement of nerves and relieves pain. For suitable candidates, an implantable interspinous spacer may provide significant benefits in terms of pain relief.

### BRIEF SUMMARY

One aspect is an interspinous spacer that includes a body having a distal portion, a proximal portion, a proximal surface, a channel extending longitudinally from the proximal surface, and at least one slot extending longitudinally along the distal portion; an arm actuator defining a threaded channel extending longitudinally, wherein the arm actuator is configured to fit within the body; an actuator screw including a shaft having a proximal end and a distal portion, wherein the actuator screw further includes a head coupled to the proximal end of the shaft, wherein the distal portion of the shaft of the actuator screw is threaded and the actuator screw is at least partially disposed in the channel of the body and the threaded channel of the arm actuator, wherein, as the actuator screw is rotated using a driver tool, the arm actuator moves longitudinally relative to the body; a first pin, wherein the first pin is arranged to move along the at least one slot of the body; a second pin; and a first arm and a second arm, wherein each of the first and second arms includes a bridge, at least two receiving extensions extending from the bridge in a first direction and configured for receiving a portion of a vertebra therebetween, and a coupling extension extending from the bridge in a second direction, wherein each of the coupling extensions defines a pin opening and a curved track, wherein the first and second arms are coupled to the distal portion of the body by the second pin extending through the curved tracks of the coupling extensions and further coupled to the distal portion of the body and the actuator arm by the first pin extending through the pin openings of the coupling extensions, wherein the first and second arms are configured to rotate among different deployment positions according to the curved track in response to longitudinal movement of the actuator arm as the actuator screw is rotated.

In at least some aspects, the actuator screw further includes a disc disposed along the shaft distal to, and separated from, the head, wherein the shaft has an outer diameter that is smaller than outer diameters of the disc and the head. In at least some aspects, the disc includes a plurality of teeth arranged around a perimeter of the disc. In at least some aspects, the interspinous spacer further includes at least one locking inset positioned within the channel of the body for engagement by the disc of the actuator screw, each of the at least one locking inset including at least one tooth for interaction with the teeth of the disc to limit rotation of the actuator screw absent the driver tool. In at least some aspects, the interspinous spacer further includes a locking ring configured for engagement with the actuator screw between the head and the disc to limit movement of the actuator screw proximally or distally within the channel of the body, wherein the body defines a bounded groove within the channel to receive the locking ring, wherein the locking ring is a partial or full ring.

In at least some aspects, the interspinous spacer further includes each of the first arm and the second arm includes at least two of the coupling extensions. In at least some aspects, the interspinous spacer further includes the coupling extensions of the first arm interleave with the coupling extensions of the second arm.

In at least some aspects, the interspinous spacer further includes the body includes opposing undercut notches configured for receiving a clamp of a spacer insertion instrument. In at least some aspects, the interspinous spacer further includes the actuator screw further includes a shaped cavity formed in the head, wherein the shaped cavity is configured for receiving a bit of the driver tool that has a shape complementary to the shaped cavity.

Another aspect is an interspinous spacer that includes a body having a distal portion, a proximal portion, a proximal surface, and a channel extending longitudinally from the proximal surface; an arm actuator defining a threaded channel extending longitudinally, wherein the arm actuator is configured to fit within the body; an actuator screw including a shaft having a proximal end and a distal portion, a head coupled to the proximal end of the shaft, and a disc disposed along the shaft distal to, and separated from, the head, wherein the distal portion of the shaft of the actuator screw is threaded and the actuator screw is at least partially disposed in the channel of the body and the threaded channel of the arm actuator, wherein the disc includes a plurality of teeth arranged around a perimeter of the disc, wherein, as the actuator screw is rotated using a driver tool, the arm actuator moves longitudinally relative to the body; at least one locking inset positioned within the channel of the body for engagement by the disc of the actuator screw, each of the at least one locking inset including at least one tooth for interaction with the teeth of the disc to limit rotation of the actuator screw absent the driver tool; and a first arm and a second arm, wherein each of the first and second arms includes a bridge, at least two receiving extensions extending from the bridge in a first direction and configured for receiving a portion of a vertebra therebetween, and a coupling extension extending from the bridge in a second direction, wherein each of the coupling extensions is coupled to the distal portion of the body and the actuator arm.

In at least some aspects, the interspinous spacer further includes a locking ring configured for engagement with the actuator screw between the head and the disc to limit movement of the actuator screw proximally or distally within the channel of the body, wherein the body defines a bounded groove within the channel to receive the locking ring, wherein the locking ring is a partial or full ring. In at least some aspects, each of the first arm and the second arm includes at least two of the coupling extensions. In at least some aspects, the coupling extensions of the first arm interleave with the coupling extensions of the second arm.

In at least some aspects, the body further includes at least one slot extending longitudinally along the distal portion of the body, the interspinous spacer further including a first pin, wherein the first pin is arranged to move along the at least one slot of the body, wherein each of the coupling extensions defines a pin opening, wherein the first and second arms are coupled to the distal portion of the body and the actuator arm by the first pin extending through the pin openings of the coupling extension. In at least some aspects, the interspinous spacer further includes a second pin, wherein each of the coupling extensions further defines a curved track, wherein the first and second arms are coupled to the distal portion of the body by the second pin through the curved tracks of the coupling extensions, wherein the first and second arms are configured to rotate relative to the body according to the curved track in response to longitudinal movement of the actuator arm as the actuator screw is rotated.

In at least some aspects, the at least one locking inset includes two locking insets disposed opposite each other. In at least some aspects, the body includes opposing undercut notches configured for receiving a clamp of a spacer insertion instrument. In at least some aspects, the actuator screw further includes a shaped cavity formed in the head, wherein the shaped cavity is configured for receiving a bit of the driver tool that has a shape complementary to the shaped cavity.

Yet another aspect is a kit that includes any of the interspinous spacers described above; a spacer insertion instrument configured to releasably grip the interspinous spacer for implantation into a patient; and the driver tool having a spacer engaging bit configured to engage the actuator screw of the interspinous spacer and rotate the actuator screw by rotation of the driver tool.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1A is a schematic perspective view of one embodiment of an interspinous spacer in a first deployed position;

FIG. 1B is a schematic perspective view of the interspinous spacer of FIG. 1A in a second deployed position;

FIG. 1C is a schematic perspective exploded view of the interspinous spacer of FIG. 1A;

FIG. 2 is schematic perspective side view of an actuator screw, locking inserts, and locking ring of the interspinous spacer of FIG. 1A;

FIG. 3 is schematic perspective side view of arms and a pin of the interspinous spacer of FIG. 1A;

FIG. 4 is schematic side view of the interspinous spacer of FIG. 1A illustrating different separation distances, represented by vertical lines, for the arms;

FIG. 5 is a perspective view of one embodiment of a spacer insertion instrument; and

FIG. 6 is a perspective view of one embodiment of a driver tool.

### DETAILED DESCRIPTION

The present invention is directed to the area of interspinous spacers for deployment between adjacent spinous processes. The present invention is also directed to systems and methods for utilizing the interspinous spacer.

Examples of interspinous spacers are found in U.S. Pat. Nos. 8,123,782; 8,128,662; 8,273,108; 8,277,488; 8,292,922; 8,425,559; 8,613,747; 8,864,828; 9,119,680; 9,155,572; 9,161,783; 9,393,055; 9,532,812; 9,572,603; 9,861,398; 9,956,011; 10,080,587; 10,166,047; 10,610,267; 10,653,456; 10,835,295; 10,835,297; 11,013,539; and 11,229,461, all of which are incorporated herein by reference. Unless indicated otherwise, the features and methods described in these references can be applied to the interspinous spacers described herein.

Conventional interspinous spacers typically have a fixed distance between the two arms when deployed. Conventionally, interspinous spacers of different sizes are available and the clinician selects which size is to be used for a particular surgery based on the size and arrangement of the vertebrae.

In addition, in at least some conventional interspinous spacers, a spindle arrangement is provided for engagement by a tool and rotation using the tool to cause the arms of the spacer to rotate for engagement with the vertebrae (for example the spinous processes of the adjacent vertebrae). The spindle arrangement is welded to the body of the interspinous spacer.

As described herein, a single interspinous spacer can have a range of deployment positions (for example, provide for a range of distances between the two arms when deployed). This allows the interspinous spacer to fit a range of different spacings between adjacent vertebrae. Additionally or alternatively, an interspinous spacer can utilize an actuator screw instead of a spindle arrangement, as described herein.

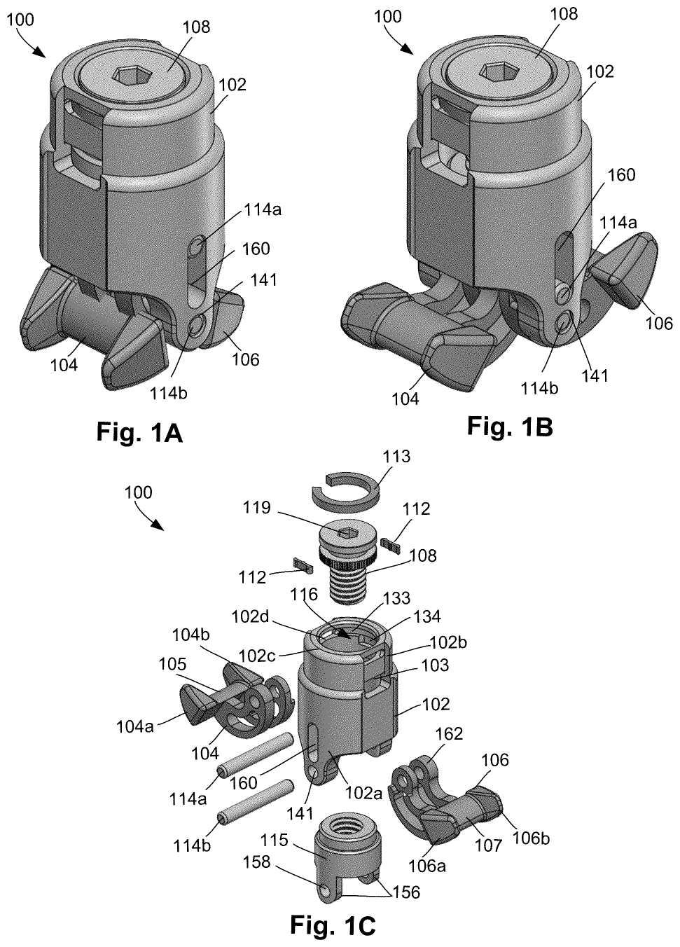

FIGS. 1A, 1B, 1C, 2, 3, and 4 illustrate one embodiment of an interspinous spacer 100 that includes a body 102, a first (or superior) arm 104, a second (or inferior) arm 106, an actuator screw 108, two locking inserts 112, a locking ring 113, a first pin 114a, a second pin 114b, and an arm actuator 115. There is no weld between the body 102 and the actuator screw 108.

In FIG. 1A, the first and second arms 104, 106 of the spacer 100 are in a first deployed position with the first and second arms 104, 106 separated by a first distance (for example, 8 mm). In FIG. 1B, the first and second arms 104, 106 of the spacer 100 are in a second deployed position with the first and second arms 104, 106 separated by a second distance (for example, 16 mm). In this embodiment, the first and second arms 104, 106 has a range of deployment positions from the first deployed position to the second deployed position. The first and second arms 104, 106 can be separated by any distance in the range from the first distance to the second distance (for example, any distance from 8 to 16 mm). This allows the spacer 100 to fit a range of different spacings between adjacent vertebrae.

The first arm 104 includes two receiving extensions 104a, 104b coupled by a bridge 105 from which an attachment portion 142 extends. The second arm 106 includes two receiving extensions 106a, 106b coupled by a bridge 107 from which the attachment portion 142 extends. In each deployment position, the pairs of receiving extensions 104a, 104b, 106a, 106b extend away from the body 102 of the spacer 100 with the extensions of each pair disposed on opposing sides of one of the adjacent vertebrae (for example, the spinous process of the adjacent vertebra), as illustrated in FIGS. 1A and 1B. The first and second arms 104, 106 of the spacer 100 are not necessarily perpendicular to the longitudinal axis of the body 102. Instead, the angle of the first and second arms 104, 106 of the spacer 100 relative to the longitudinal axis of the body 102 depends on the selected deployed position which can range from the first deployed position of FIG. 1A to the second deployed position of FIG. 1B. In at least some embodiments, the shape of the bridges 105, 107 is selected to provide suitable engagement of the adjacent vertebrae for the range of selectable deployed positions.

In at least some embodiments, the length of the bridge 105 of the first arm 104 is approximately 7 to 10 millimeters and the length of the bridge 107 of the second arm 106 is approximately 5 to 8 millimeters. In at least some embodiments, the tip-to-tip distance of the extensions 104a, 104b is approximately 8 to 12 millimeters and the tip-to-tip distance of the extensions 106a, 106b is approximately 8 to 12 millimeters. In at least some embodiments, the first arm 104 forms a larger space for receiving the superior vertebra (for example, the superior spinous process) than the space formed by the second arm 106 for receiving the inferior vertebra (for example, the inferior spinous process) as vertebrae and spinous processes are naturally narrower on top and wider on the bottom. In at least some embodiments, where there is a difference in size between the first and second arms 104, 106, the spacer 100 may include a marking or other indication so that a clinician can individually identify the first and second arms 104, 106 for correct implantation orientation within the patient.

In at least some other embodiments, the first and second arms 104, 106 form a same-sized space for receiving the vertebrae. In at least some embodiments, the bridges 105, 107 of the first and second arms 104, 106 have a same length.

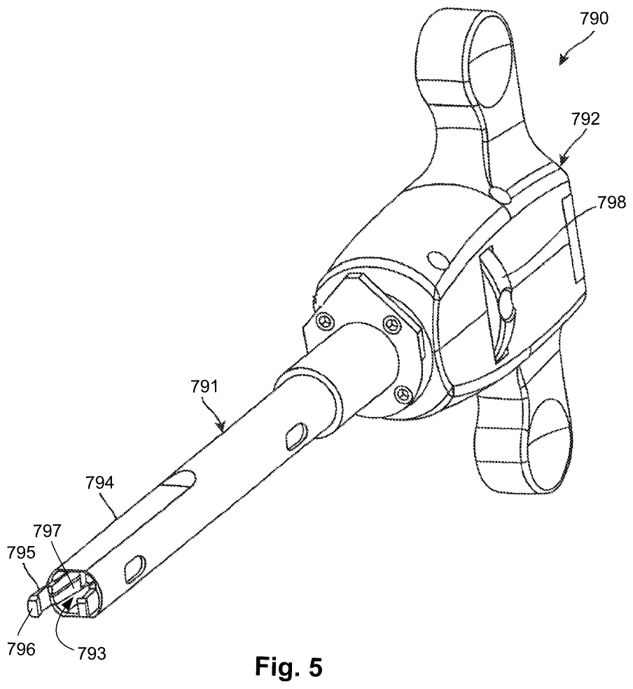

The body 102 includes a distal portion 102a, a proximal portion 102b, a proximal surface 102c, and an opening 102d in the proximal surface for the actuator screw 108. The body 102 defines a channel 116 that extends distally from the opening 102d through at least a portion of the body 102, as illustrated in FIG. 1C. In at least some embodiments, the body 102 includes undercut notches 103 formed on opposite sides of the proximal portion 102b of the body. In at least some embodiments, the notches 103 are configured for attachment of clamps 795 of a spacer insertion instrument 790 (FIG. 5).

The body 102 includes opposing slots 160 (or at least one slot) for receiving a first pin 114a and travel of the first pin along the slots as the first and second arms 104, 106 are deployed or retracted. The body 102 also includes opposing pin holes 141, distal of the opposing slots 160, for receiving a second pin 114b.

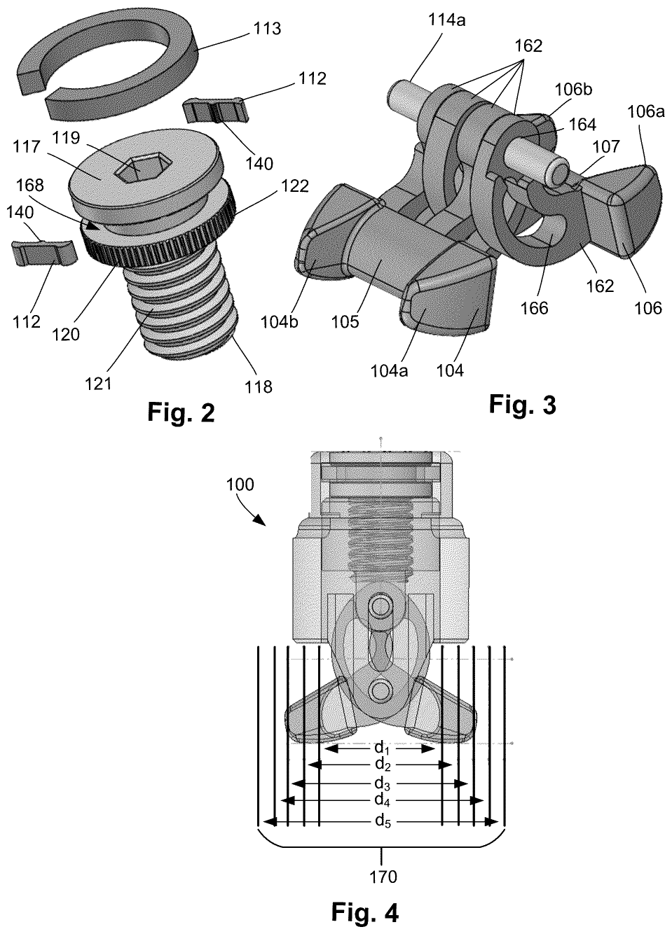

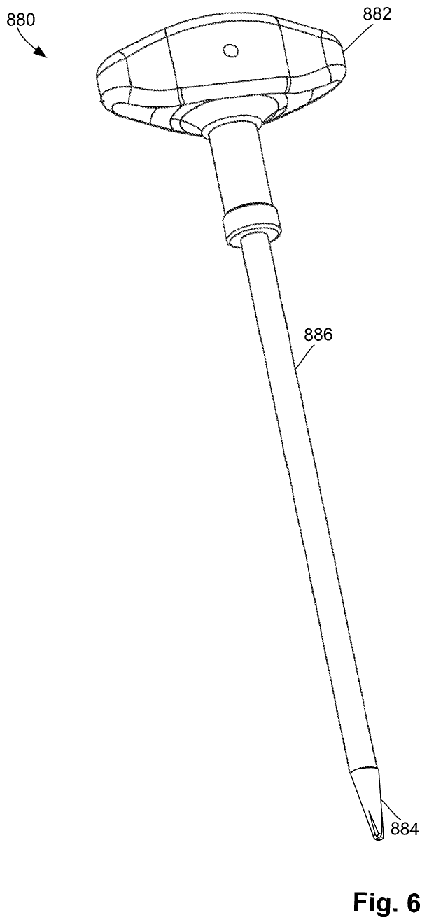

The actuator screw 108 includes a head 117, a shaft 118, and a disc 120 disposed along the shaft and having teeth 122 arranged around the perimeter of the disc, as illustrated in FIG. 2. The disc 120 has a larger outer diameter than the shaft 118 and is positioned distal to the head 117 with a gap 168 between the disc and the head. At least a portion 121 of the shaft 118 distal to the disc is threaded. The actuator screw 108 can be made from a single piece of material or may contain two or more components that are attached together. The head 117 of the actuator screw 108 includes a shaped cavity 119 to receive a driver tool 880 (FIG. 6) with a complementary-shaped engaging bit 884. Engagement of the actuator screw 108 by the driver tool allows a user to rotate the actuator screw to further separate (or, in at least some embodiments, retract) the first and second arms 104, 106.

A locking ring 113 fits on the actuator screw 108 in the gap 168 between the head 117 and the disc 120. As the actuator screw 108 is inserted into the channel 116 of the body 102, the locking ring 113 fits within a bounded groove 133 in the interior wall of the body. The locking ring 113 resists movement of the actuator screw 108 up or down (e.g., distally or proximally) within the body 102. The locking ring 113 can be a full ring or a partial ring (as illustrated in FIG. 1C).

Locking insets 112 fit within opposing indents 134 located below the bounded groove 133 in the interior wall of the body 102 so that the locking insets 112 are exposed within the body. Each locking inset 112 includes a body 138 and at least one tooth 140 (FIG. 2) extending from the body. The at least one tooth 140 of the locking inset 112 is arranged to engage the teeth 122 of the disc 120 of the actuator screw 108 when the actuator screw is disposed within the body 102. The at least one tooth 140 of the locking insets 112 and the teeth 122 of the disc 120 of the actuator screw 108 are arranged to resist rotation of the actuator screw except by use of a tool 880 (FIG. 6) that engages the actuator screw 108. In at least some embodiments, the shape and size of the at least one tooth 140 of the locking insets 112 and the teeth 122 of the disc 120 of the actuator screw 108 are selected to resist rotation of the actuator screw when the first and second arms 104, 106 are separated at a selected deployment position and force is applied to the first and second arms 104, 106 such as, for example, during the patient's movement and bending. In at least some embodiments, the shape and size of the at least one tooth 140 of the locking insets 112 and the teeth 122 of the disc 120 of the actuator screw 108 are selected to generate a clicking sound as the actuator screw 108 is rotated using a tool 880 (FIG. 6).

The arm actuator 115 includes a threaded channel 154 into which the threaded portion 121 of the shaft 118 of the actuator screw 108 extends. The threads and the size of the threaded channel 154 of the arm actuator 115 and the threaded shaft 118 of the actuator screw 108 are complementary so that the actuator screw 108 fits within the threaded channel 154 and moves distally or proximally, along a path defined by the threads, as the actuator screw 108 is rotated. The arm actuator 115 further includes two opposing actuator extensions 156 that each define a pin opening 158 for receiving the first pin 114a.

Each of the first and second arms 104, 106 includes at least one coupling extension 162 extending from the bridge 105, 107. Each coupling extension 162 defines an opening 164 for receiving the first pin 114a, as illustrated in FIG. 3, and a curved track 166 for receiving the second pin 114b and allowing the second pin to move along the curved track in response to rotation of the actuator screw 108. In the illustrated embodiment of FIGS. 1A, 1B, 1C, and 3, each of the first and second arms 104, 106 includes two coupling extensions 162 that, when the spacer 100 is assembled, interleave with each other as illustrated in FIG. 3. When the spacer 100 is assembled, the first pin 114a passes through the opposing slots 160 of the body 102, the pin openings 158 of the arm actuator 115, and the openings 164 of the coupling extensions 162 of the first and second arms 104, 106. The second pin 115b passes through the opposing pin holes 141 of the body 102 and the curved tracks 166 of the coupling extensions 162 of the first and second arms 104, 106.

As the actuator screw 108 is rotated in a first direction, the arm actuator 115 moves distally. The first pin 114a is carried distally by the arm actuator 115 pushing the portions of the first and second arms 104, 106 adjacent to the first pin 114a distally.

This causes the first and second arms 104, 106 to rotate about the second pin 114b according to the path of the curved tracks 166 of the first and second arms 104, 106 resulting in the first and second arms separating from each other, as illustrated by comparing FIGS. 1A and 1B.

Rotating the actuator screw 108 in a second direction, opposite the first direction, reverses the movement of the arm actuator 115, pin 114a, and first and second arms 104, 106. The ends of the opposing slots 160 of the body 102 limit movement of the first pin 114a and, thereby, limit the range of separation of the first and second arms 104, 106.

In the first deployed position of FIG. 1A, the first pin 114a is at the most proximal position along opposing slots 160 in the body 102. In the first deployed position of FIG. 1B, the first pin 114a is at the most distal position along the opposing slots 160 in the body 102. FIG. 4 illustrates examples of different separation distances 170 (representing different deployed positions) between the first and second arms 104, 106 for one embodiment of the spacer 100. Examples of separation distances are illustrated in FIG. 4 as d₁, d₂, d₃, d₄, and ds (for example, 8, 10, 12, 14, or 16 mm, respectively). In at least some embodiments, any distance between the largest and smallest separation distance can be achieved. In at least some embodiments, the selectable distances may be defined in part by the teeth 122 on the disc 120 of the actuator screw 118, as well as the length of the opposing slots 160 of the body 102.

U.S. Pat. Nos. 8,123,782; 8,128,662; 8,273,108; 8,277,488; 8,292,922; 8,425,559; 8,613,747; 8,864,828; 8,945,183; 9,119,680; 9,155,572; 9,161,783; 9,393,055; 9,532,812; 9,572,603; 9,861,398; 9,956,011; 10,080,587; 10,166,047; 10,610,267; 10,653,456; 10,835,295; 10,835,297; 11,013,539; and 11,229,461, all of which are incorporated herein by reference, illustrate a variety of tools for insertion and deployment of a spacer between adjacent spinous processes. These tools can be used or modified for insertion and deployment of the spacer 100 described above.

As an example, FIGS. 5 and 6 illustrate a spacer insertion instrument 790 and a driver tool 880, respectively. The spacer insertion instrument 790 includes a cannula 791 connected to a handle 792. The spacer insertion instrument 790 defines a central passageway 793 through the handle 792 and cannula 791. The driver tool 880 is removably insertable into the central passageway 793.

The cannula 791 includes clamps (for example, prongs) 795 to releasably clamp to the body 102 of the spacer 100 (for example, to the undercut notches 103 formed on opposite sides of the body 102) for delivery of the spacer into the patient using the spacer insertion instrument 790. In at least some embodiments, the clamps 795 include extensions 796 that extend inwardly toward each other to form hooks. In at least some embodiments, the extensions 796 can engage the undercut notches 103 (FIG. 1C) formed on opposite sides of the body 102 of the spacer 100 to grip the spacer.

The cannula 791 also includes an inner shaft 797 (to which the clamps 795 are attached), an outer shaft 794, and a control 798. In at least some embodiments, the inner shaft 797 is connected to the handle 792 and the outer shaft 794 is passed over the inner shaft 797.

The outer shaft 794 translates with respect to the inner shaft 797 (or, alternatively, the inner shaft translates with respect to the outer shaft) using the control 798. The translation of the outer shaft 794 (or the inner shaft 797) operates the clamps 795. When the outer shaft 794 moves away from the clamps 795, the clamps separate to allow loading (or unloading) of the spacer 100 on the spacer insertion instrument 790. When the outer shaft 794 moves toward the clamps 795, the clamps are moved together to grip the spacer 100. For example, the clamps 795 can grip the undercut notches 103 formed on opposite sides of the body 102 of the spacer 100. In this manner, the spacer insertion instrument 790 can hold the spacer 100 for delivery of the spacer into position between adjacent spinous processes within the patient.

Turning to FIG. 8, a driver tool 880 includes a handle 882 at the proximal end and a spacer engaging bit 884 (for example, a socket key or hexagonal tip) at the distal end. The handle 882 and spacer engaging bit 884 are connected by a shaft 886. The driver tool 880 is sized to be inserted into the central passageway 793 of the spacer insertion instrument 790 such that the spacer engaging bit 884 at the distal end operatively connects with a spacer 100 gripped by the clamps 795 of the spacer insertion instrument 790. The spacer engaging bit 884 includes features for engaging with the shaped cavity 119 (see, for example. FIG. 2) in the actuator screw 108 of the spacer 100. In at least some embodiments, the driver tool 880 has a spacer engaging bit 884 that is complementary to the shaped cavity 119 in the actuator screw 108 of the spacer 100. For example, the bit 884 can have a flat (like a regular screwdriver), cross (like a Phillips screwdriver), square, pentagonal, hexagonal, or octagonal shape (or any other suitable shape) with the shaped cavity 119 having a complementary shape. Rotating the driver tool 880 when engaged with the actuator screw 108 of the spacer 100 rotates the actuator screw 108 to separate the arms 104, 106 of the spacer, as described above.

In at least some embodiments, a small midline or lateral-to-midline incision is made in the patient for percutaneous delivery of the spacer 100. In at least some embodiments, the supraspinous ligament is avoided. In at least some embodiments, the supraspinous ligament is split longitudinally along the direction of the tissue fibers to create an opening for the instrument. In at least some embodiments, one or more dilators may be used to create or enlarge the opening.

In at least some embodiments, the spacer 100, in the first deployed position, is releasably attached to the spacer insertion instrument 790 as described above. In at least some embodiments, the spacer 100 is inserted into a port or cannula, if one is employed, which has been operatively positioned to form an opening to the interspinous space within a patient's back. The spacer 100, attached to the spacer insertion instrument 790, is inserted into the interspinous space between the spinous processes of two adjacent vertebral bodies. In at least some embodiments, the spacer 100 is advanced beyond the end of a cannula or, alternatively, the cannula is pulled proximately to uncover the spacer 100 connected to the spacer insertion instrument 790. Once in position, the driver tool 880 is inserted into the spacer insertion instrument 790, if not previously inserted, to engage the actuator screw 108. The driver tool 880 is rotated to rotate the actuator screw 108. The rotating actuator screw 108 changes the deployed position of the spacer 100. Rotation in one direction, for example, clockwise, for example, increase the separation distance between the arms 104, 106 (compare, for example, FIGS. 1A and 1B).

The arms 104, 106 of the spacer may be positioned in one of many deployed positions with different separation distances. In at least some, embodiments, the separation of the arms 104, 106 can be reversed by rotating the actuator screw 108 in the opposite direction, for example, counterclockwise.

In at least some embodiments, a clinician can observe with fluoroscopy or other imaging technique the positioning of the spacer 100 inside the patient and then choose to reposition the spacer 100, if desired. Repositioning of the spacer may involve reversing, or partially reversing, the separation of the arms 104, 106. The spacer 100 may then be re-deployed into the desired location. This process can be repeated as necessary until the clinician has achieved the desired positioning of the spacer in the patient.

Following deployment of the spacer, the spacer insertion instrument 790 and driver tool 880 (and any other instrumentation, such as a cannula or dilator) is removed from the body of the patient. The spacer insertion instrument 790 can be operated as described above to release the clamps 795 from the spacer 100.

The above specification provides a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. An interspinous spacer, comprising:
a body having a distal portion, a proximal portion, a proximal surface, a channel extending longitudinally from the proximal surface, and at least one slot extending longitudinally along the distal portion;
an arm actuator defining a threaded channel extending longitudinally, wherein the arm actuator is configured to fit within the body;
an actuator screw comprising a shaft having a proximal end and a distal portion, wherein the actuator screw further comprises a head coupled to the proximal end of the shaft, wherein the distal portion of the shaft of the actuator screw is threaded and the actuator screw is at least partially disposed in the channel of the body and the threaded channel of the arm actuator, wherein, as the actuator screw is rotated using a driver tool, the arm actuator moves longitudinally relative to the body;
a first pin, wherein the first pin is arranged to move along the at least one slot of the body;
a second pin; and
a first arm and a second arm, wherein each of the first and second arms comprises a bridge, at least two receiving extensions extending from the bridge in a first direction and configured for receiving a portion of a vertebra therebetween, and a coupling extension extending from the bridge in a second direction, wherein each of the coupling extensions defines a pin opening and a curved track, wherein the first and second arms are coupled to the distal portion of the body by the second pin extending through the curved tracks of the coupling extensions and further coupled to the distal portion of the body and the actuator arm by the first pin extending through the pin openings of the coupling extensions, wherein the first and second arms are configured to rotate among different deployment positions according to the curved track in response to longitudinal movement of the actuator arm as the actuator screw is rotated.

2. The interspinous spacer of claim 1, wherein the actuator screw further comprises a disc disposed along the shaft distal to, and separated from, the head, wherein the shaft has an outer diameter that is smaller than outer diameters of the disc and the head.

3. The interspinous spacer of claim 2, wherein the disc comprises a plurality of teeth arranged around a perimeter of the disc.

4. The interspinous spacer of claim 3, further comprising at least one locking inset positioned within the channel of the body for engagement by the disc of the actuator screw, each of the at least one locking inset comprising at least one tooth for interaction with the teeth of the disc to limit rotation of the actuator screw absent the driver tool.

5. The interspinous spacer of claim 3, further comprising a locking ring configured for engagement with the actuator screw between the head and the disc to limit movement of the actuator screw proximally or distally within the channel of the body, wherein the body defines a bounded groove within the channel to receive the locking ring, wherein the locking ring is a partial or full ring.

6. The interspinous spacer of claim 1, wherein each of the first arm and the second arm comprises at least two of the coupling extensions.

7. The interspinous spacer of claim 6, wherein the coupling extensions of the first arm interleave with the coupling extensions of the second arm.

8. The interspinous spacer of claim 1, wherein the body comprises opposing undercut notches configured for receiving a clamp of a spacer insertion instrument.

9. The interspinous spacer of claim 1, wherein the actuator screw further comprises a shaped cavity formed in the head, wherein the shaped cavity is configured for receiving a bit of the driver tool that has a shape complementary to the shaped cavity.

10. A kit, comprising:
the interspinous spacer of claim 1;
a spacer insertion instrument configured to releasably grip the interspinous spacer for implantation into a patient; and
the driver tool comprising a spacer engaging bit configured to engage the actuator screw of the interspinous spacer and rotate the actuator screw by rotation of the driver tool.

11. An interspinous spacer, comprising:
a body having a distal portion, a proximal portion, a proximal surface, and a channel extending longitudinally from the proximal surface;
an arm actuator defining a threaded channel extending longitudinally, wherein the arm actuator is configured to fit within the body;
an actuator screw comprising a shaft having a proximal end and a distal portion, a head coupled to the proximal end of the shaft, and a disc disposed along the shaft distal to, and separated from, the head, wherein the distal portion of the shaft of the actuator screw is threaded and the actuator screw is at least partially disposed in the channel of the body and the threaded channel of the arm actuator, wherein the disc comprises a plurality of teeth arranged around a perimeter of the disc, wherein, as the actuator screw is rotated using a driver tool, the arm actuator moves longitudinally relative to the body;
at least one locking inset positioned within the channel of the body for engagement by the disc of the actuator screw, each of the at least one locking inset comprising at least one tooth for interaction with the teeth of the disc to limit rotation of the actuator screw absent the driver tool; and
a first arm and a second arm, wherein each of the first and second arms comprises a bridge, at least two receiving extensions extending from the bridge in a first direction and configured for receiving a portion of a vertebra therebetween, and a coupling extension extending from the bridge in a second direction, wherein each of the coupling extensions is coupled to the distal portion of the body and the actuator arm.

12. The interspinous spacer of claim 11, further comprising a locking ring configured for engagement with the actuator screw between the head and the disc to limit movement of the actuator screw proximally or distally within the channel of the body, wherein the body defines a bounded groove within the channel to receive the locking ring, wherein the locking ring is a partial or full ring.

13. The interspinous spacer of claim 11, wherein each of the first arm and the second arm comprises at least two of the coupling extensions.

14. The interspinous spacer of claim 13, wherein the coupling extensions of the first arm interleave with the coupling extensions of the second arm.

15. The interspinous spacer of claim 11, wherein the body further comprises at least one slot extending longitudinally along the distal portion of the body, the interspinous spacer further comprising a first pin, wherein the first pin is arranged to move along the at least one slot of the body, wherein each of the coupling extensions defines a pin opening, wherein the first and second arms are coupled to the distal portion of the body and the actuator arm by the first pin extending through the pin openings of the coupling extension.

16. The interspinous spacer of claim 15, further comprising a second pin, wherein each of the coupling extensions further defines a curved track, wherein the first and second arms are coupled to the distal portion of the body by the second pin through the curved tracks of the coupling extensions, wherein the first and second arms are configured to rotate relative to the body according to the curved track in response to longitudinal movement of the actuator arm as the actuator screw is rotated.

17. The interspinous spacer of claim 11, wherein the at least one locking inset comprises two locking insets disposed opposite each other.

18. The interspinous spacer of claim 11, wherein the body comprises opposing undercut notches configured for receiving a clamp of a spacer insertion instrument.

19. The interspinous spacer of claim 11, wherein the actuator screw further comprises a shaped cavity formed in the head, wherein the shaped cavity is configured for receiving a bit of the driver tool that has a shape complementary to the shaped cavity.

20. A kit, comprising:
the interspinous spacer of claim 11;
a spacer insertion instrument configured to releasably grip the interspinous spacer for implantation into a patient; and
the driver tool comprising a spacer engaging bit configured to engage the actuator screw of the interspinous spacer and rotate the actuator screw by rotation of the driver tool.
